(19) United States
(12) Patent Application Publication
(10) Pub. No.: US 2003/0001951 A1
(43) Pub. Date: Jan. 2, 2003

(54) Image obtaining method and apparatus for an endoscope

(75) Inventors: Kazuhiro Tsujita, Kaisei-machi (JP); Tomonari Sendai, Kaisei-machi (JP)

Correspondence Address: SUGHRUE MION, PLLC, 2100 PENNSYLVANIA AVENUE, N.W., WASHINGTON, DC, 20037, US

(73) Assignee: FUJI PHOTO FILM CO., LTD.

(21) Appl. No.: 10/179,396

(22) Filed: Jun. 26, 2002

(30) Foreign Application Priority Data
Jun. 27, 2001 (JP) 194150/2001

(51) Int. Cl. H04N 9/47

(52) U.S. Cl. 348/65; 377/60; 348/251

(57) ABSTRACT

An image obtaining method and apparatus for an endoscope, wherein the S/N ratio of an observation image formed from the image signal obtained by a solid state image obtaining element equipped with a charge multiplying shift resistor is improved. A signal charge representing a fluorescent image is obtained of the fluorescent image formed of the fluorescent light emitted from a target subject upon the irradiation thereof by an excitation light emitted from an illuminating means, by a charge multiplying shift resistor. A readout means reads out the signal charge and outputs an image signal based on the read out signal charge. In obtaining an observation image signal based on the read out image signal, a computing means subtracts the dark noise image signal component from the read out image signal to obtain the observation image signal.

### BACKGROUND OF THE INVENTION

1. Field of the Invention

The present invention relates to an image obtaining method and apparatus for an endoscope, and in particular to an image obtaining method and apparatus, which employs a solid state image obtaining element having charge multiplying type shift resistor utilizing an impact ionization phenomenon, for an endoscope.

2. Description of the Related Art

In general, the noise component included in an image signal obtained by the read out of a signal charge from a solid state image obtaining element such as a CCD or the like is formed of read noise and dark noise. In a case, for example, wherein, utilizing an endoscope, a living tissue (hereinafter referred to as a target subject) within a body cavity is to be observed as a motion picture at a frame rate of 60 frames/second, an image formed of extremely weak light is detected by a solid state image obtaining element and obtained thereby as a signal charge, and the observation image signal formed based on the image signal obtained by the read out of the aforementioned signal charge from the solid state image obtaining element is used as the aforementioned motion picture that is to be observed; however, there are cases in which the aforementioned noise component deteriorates the quality of the observation image signal.

However, charge multiplying type solid state image obtaining elements utilizing an impact ionization phenomenon to amplify the charge (hereinafter referred to as charge multiplying shift resistor equipped solid state image obtaining elements) have been developed. It is known that if a charge multiplying shift resistor equipped solid state image obtaining element is used, the photoelectrically converted signal charge can be amplified without incurring an increase in the read noise. Investigations as to whether or not the read noise can be reduced and the S/N ratio of the obtained image signal can be improved by utilizing an endoscope provided with a charge multiplying shift resistor equipped solid state image obtaining element in the obtainment of the motion picture of the target subject within a body cavity by use of, are progressing.

Note that the charge multiplying shift resistor equipped solid state image obtaining element is an image obtaining element provided with a shift resistor having a charge multiplying function, which is disposed between the horizontal readout resistor of a CCD and a output amplifier; the photoelectrically converted signal charge is amplified by the impact ionization effect occurring within the aforementioned shift resistor. The principle by which the shift resistor provided with a charge multiplying function multiplies the signal charge employs an impact ionization phenomenon (the phenomenon whereby positive holes are formed when electrons collide with the silicon elements forming the shift resistor), wherein secondary electrons are produced when a signal charge is transmitted into a deep potential formed by an ample intensity. By transmitting, by use of multi-step shift resistors each of which have a deep potential, the signal charge that has been transmitted from the horizontal shift resistor, the aforementioned two-dimensional electrons can be produced repeatedly, and the signal charge can be amplified without incurring an amplification of the read noise. A detailed description of this type of charge multiplying shift resistor equipped solid state image obtaining element may be found in the specification of U.S. Pat. No. 5,337,340.

However, the charge multiplying shift resistor equipped solid state image obtaining element described above exhibits the same performance characteristics with respect to dark noise as the conventional solid state image obtaining element, and if configured so as to suppress the generation of read noise, the problem of the dark noise included in the image signal obtained by the read out of the signal charge obtained by the charge multiplying shift resistor equipped solid state image obtaining element becomes conspicuous. In particular, with regard to an endoscope apparatus that is required to have a performance level enabling the accurate observation of an image formed of extremely faint light composed of a few photons such as is the case in which the fluorescent light emitted from a target subject within a body cavity is obtained as an image, it is even more desirable that the dark noise that becomes mixed with the observation image signal is suppressed.

### SUMMARY OF THE INVENTION

The present invention has been developed in consideration of the forgoing circumstances, and it is an objective of the present invention to provide an image obtaining method and apparatus for an endoscope, wherein the S/N ratio of an observation image signal formed from the image signal obtained by a charge multiplying shift resistor equipped solid state image obtaining element can be improved in comparison to the S/N ratio of an observation image signal formed from the image signal obtained by a conventional solid state image obtaining elements.

The image obtaining method, which is for application in an endoscope, according to the present invention comprises performing the steps of: obtaining, by use of a charge multiplying shift resistor equipped solid state image obtaining element, an image formed of the light emitted or reflected from a target subject upon the irradiation thereof by a light, and a signal charge representing the obtained image; reading out, by use of a readout means, said signal charge and outputting an image signal based on the read out signal charge; and obtaining an observation image signal based on the outputted image signal; wherein, the dark noise image signal component representing the dark noise included within the image signal read out by the readout means is subtracted from said image signal to obtain the observation image signal.

The image obtaining apparatus according to the present invention comprises: an illuminating means for projecting a light onto a target subject; a charge multiplying shift resistor equipped solid state image obtaining element for obtaining an image formed of the light emitted or reflected from a target subject upon the irradiation thereof by the aforementioned light, and a signal charge representing the obtained image; and a readout means for reading out said signal charge from the charge multiplying shift resistor equipped solid state image obtaining element and outputting an image signal based on the read out signal charge; further comprising a subtracting means for subtracting the dark noise image signal component representing the dark noise included within the image signal read out by the readout means from said image signal to obtain a subtraction image signal.

The dark noise image signal component may be the image signal read out from the charge multiplying shift resistor equipped solid state image obtaining element by the readout means when the light is not being emitted.

The dark noise image signal component may be obtained in advance and prerecorded in the subtraction means.

The image obtaining apparatus for an endoscope may further comprise an addition averaging means for adding and averaging (hereinafter referred to as addition averaging) a plurality of subtraction image signals successively outputted from the subtraction means to obtain an addition averaged subtraction image signal.

The readout means can be a readout means that reads out the signal charge at a frame rate greater than 60 frames/sec.

The expression “light emitted from the target subject” refers to the autofluorescence light or the like emitted from the target subject upon the irradiation thereof by a light having a wavelength on the shorter wavelength side of the visible wavelength range.

The referents of “light reflected from the target subject” include, the light reflected from the target subject upon the irradiation thereof by a reference light referred to in order to determine the intensity of the light projected onto the target subject such as a near-infrared light, the light reflected from the target subject upon the irradiation thereof by an observation light for performing standard observation such as white light, etc.

The referents of “observation image signal” include the signal representing a visible rendition of the target subject, a signal representing the results of an analysis of an image signal, etc.

According to the image obtaining method and apparatus of the present invention: in obtaining an observation image signal based on the obtainment of an image of the target subject by the charge multiplying shift resistor equipped solid state image obtaining element, because the dark noise image signal component representing the dark noise included in the image signal read out by the readout means has been subtracted from said read out image signal to obtain a subtraction image based upon which the observation image signal is formed, the quantity of dark noise contained in the subtraction image is reduced, and the S/N ratio of the observation image signal formed based on said subtraction image can be improved in comparison to that obtained by conventional means.

Further, if the dark noise image signal component is the image signal read out from the charge multiplying shift resistor equipped solid state image obtaining element by the readout means when the light is not being emitted, and is obtained in advance and prerecorded in the subtraction means, the dark noise image signal component can be more accurately subtracted from the image signal, and the S/N ratio of the observation image signal formed based on said subtraction image can be improved in comparison to that obtained by conventional means.

Still further, if an addition averaging means for adding and averaging a plurality of subtraction image signals successively outputted from the subtraction means to obtain an addition averaged subtraction image is provided, the margin of error of the dark noise image signal component can be averaged, and the S/N ratio of the observation image signal formed based on said subtraction image can be improved in comparison to that obtained by conventional means.

In addition, if the readout means is a readout means that reads out the signal charge at a frame rate greater than 60 frames/sec, the signal charge can be read out at a faster speed than that read out from a charge multiplying shift resistor equipped solid state image obtaining element at the conventional 60 frames/sec frame rate, and the quantity of the dark noise component included in the image signal obtained by reading out the aforementioned signal charge can be reduced in comparison to that obtained by conventional means. This is due to the fact that as the signal charge readout speed of a solid state image obtaining element (i.e., conventional solid state image obtaining elements and charge multiplying shift resistor equipped solid state image obtaining elements generally available on the consumer market) becomes faster, the quantity of dark noise accumulated thereon is reduced.

### BRIEF DESCRIPTION OF THE DRAWINGS

FIG. 1 is a block diagram of the main part of the fluorescent endoscope apparatus according to an embodiment of the present invention,

FIG. 2 is a timing chart showing the timing at which the signal charge is read out by the readout means,

FIG. 3 is a block drawing of a fluorescent endoscope apparatus that has been further provided with an addition averaging means,

FIG. 4 is a timing chart showing the timing at which the signal charge is read out by the readout means,

FIG. 5 is a block drawing of a fluorescent endoscope apparatus that has been further provided with a normalized fluorescent light intensity computing means,

FIG. 6 is a timing chart showing the timing at which the signal charge is read out by the readout means, and

FIG. 7 is a schematic drawing of the configuration of a mosaic filter.

### DESCRIPTION OF THE PREFERRED EMBODIMENTS

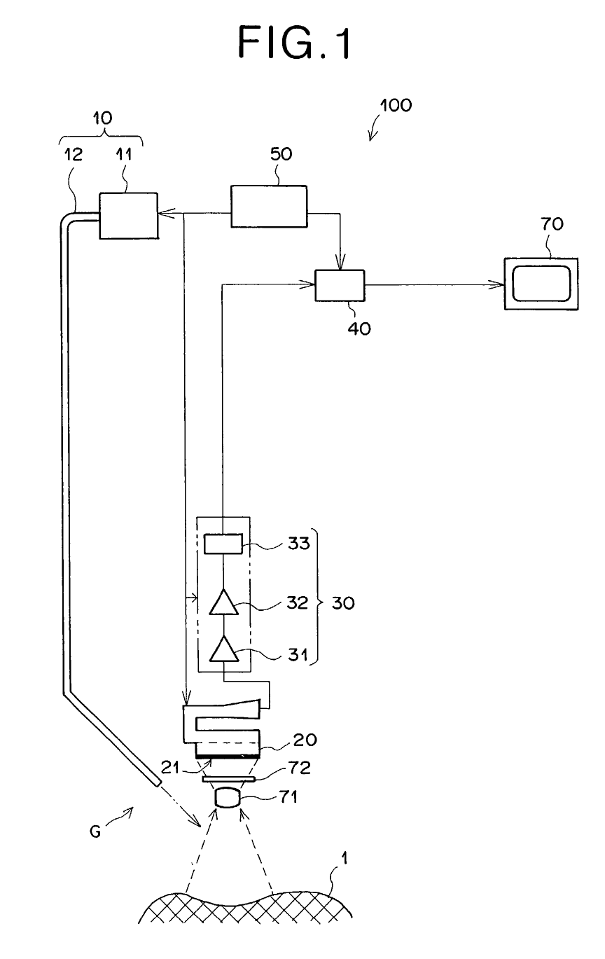

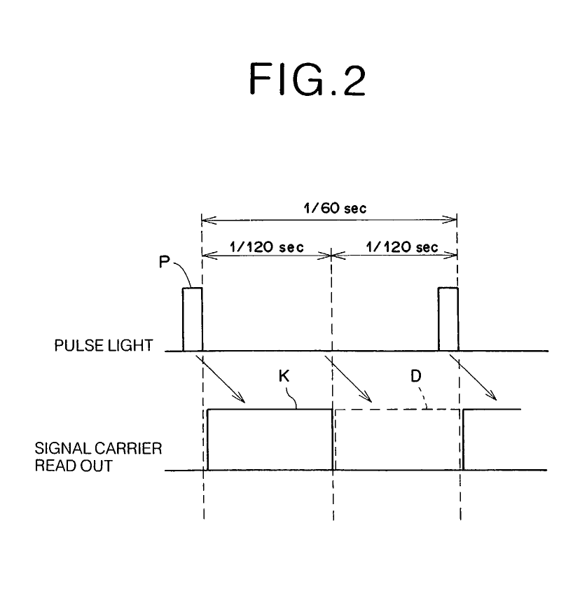

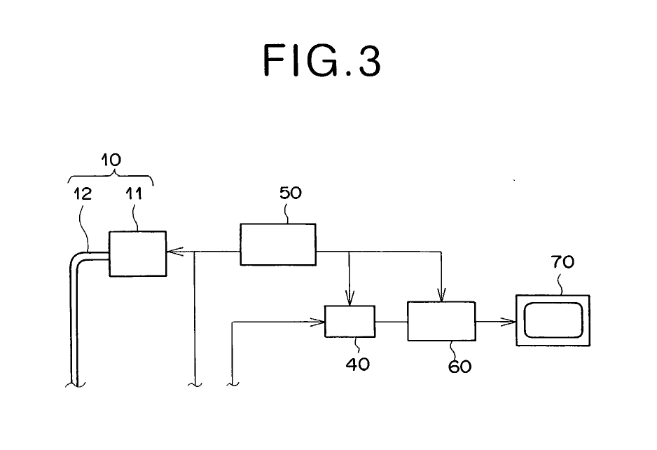

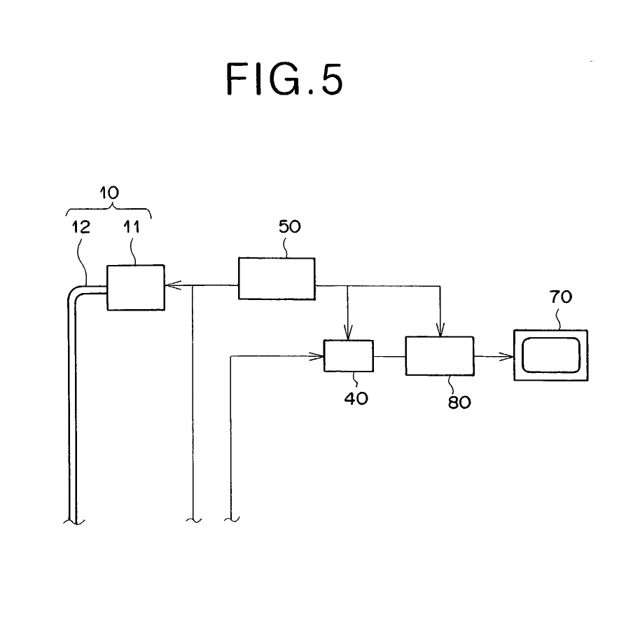

Hereinafter a preferred embodiment of the present invention will be explained with reference to the attached drawings. FIG. 1 is a block diagram showing the structure of the image obtaining apparatus, which is for application in an endoscope, according to an embodiment of the present invention. FIGS. 2, 4, and 6 are timing charts showing the timing at which the signal charge is read out by the readout means. FIG. 3 is a block drawing of the fluorescent endoscope apparatus of FIG. 1 that has been further provided with an addition averaging means. FIG. 5 is a block drawing of the fluorescent endoscope apparatus of FIG. 1 that has been further provided with a normalized fluorescent light intensity computing means

The fluorescent endoscope 100 comprises: an illuminating means 10 for projecting a light onto a target subject 1, which is a living tissue within a body cavity of a patient; a charge multiplying shift resistor equipped solid state image obtaining element 20 for obtaining an image formed of the light emitted from the target subject 1 or reflected from the target subject 1 upon the irradiation thereof by the aforementioned light, and a signal charge representing the obtained image; a readout means 30 for reading out the signal charge from the charge multiplying shift resistor equipped solid state image obtaining element 20 and outputting an image signal based on the read out signal charge; a subtracting means 40 for subtracting the dark noise image signal component representing the dark noise included within the image signal readout by the readout means 30 from said image signal to obtain a subtraction image signal; a display 70 for converting the subtraction image signal, which is an observation image signal, outputted from the subtracting means 40 to a visible image signal and displaying said visible image signal; and a controller 50 for controlling the operation and operational timing of the illuminating means 10, the charge multiplying shift resistor equipped solid state image obtaining element 20, the readout means 30, and the subtracting means 40, etc.

The illuminating means 10 comprises: a light source apparatus 11 provided with a Gallium nitride type pulse drive semiconductor laser (GaN-LD) for projecting a first light, which is a pulse type excitation light of a wavelength near 410 nm, onto the target subject 1 so as to cause the emission of fluorescent light therefrom, and a pulse drive laser for projecting a second light, which is a pulse type infrared light that serves as a reference light, onto the target subject 1; and a light guide 12 for guiding the excitation light and the near infrared light emitted from the light source apparatus 11 to the distal end portion G of the endoscope.

The readout means 30 comprises: a floating diffusion amplifier 31 for converting the signal charges outputted from the charge multiplying shift resistor equipped solid state image obtaining element 20 to voltages; an A/D conversion gain adjusting amplifier 32 for amplifying the output of the floating diffusion amplifier 31; and an A/D convertor 33 for converting the output of the A/D conversion gain adjusting amplifier 32 to an image signal, which is formed of digital values.

Next, the operation of the above-described embodiment will be explained.

The pulse type excitation light emitted from the light source apparatus 11 passes through the light guide 12 and is projected onto the target subject 1. As shown in the timing chart of FIG. 2, the fluorescent light emitted from the target subject 1 upon the irradiation thereof by the pulse type excitation light P passes through the fluorescent light focusing lens 71 and is focused onto the light detecting surface 21 of the charge multiplying shift resistor equipped solid state image obtaining element 20 (hereinafter referred to as a DZS 20). Note that an excitation light cutoff filter 72 is disposed between the focusing lens 71 and the light detecting surface 21; wherein, the excitation light directed toward the light detecting surface 21 together with the fluorescent light is cutoff by said excitation light cutoff filter 72. The DZS 20 detects the fluorescent image focused on the light detecting surface 21, and obtains a signal charge representing said fluorescent image. The fluorescent image signal charge obtained by the DZS 20 is read out by the readout means 30 at a frame rate of 120 frames/sec, converted to a fluorescent image signal formed of digital values, outputted, and recorded in the subtraction means 40. Note that the frame rate at which the aforementioned signal charge is read out is represented by a value 1/(image exposure time+readout time of the exposed image).

Continuing, as shown in the timing chart of FIG. 2, an image of the target subject 1 not irradiated by the excitation light is obtained by the DZS 20 while the excitation light is not being emitted, that is, the dark noise signal charge component is obtained 1/120 seconds after the fluorescent image is obtained. The dark noise image signal charge obtained by the DZS 20 is read out by the readout means 30 at the same frame rate of 120 frames/sec, converted to a dark noise image signal component representing the dark noise, outputted, and recorded in the subtraction means 40.

Here, the readout time of the reading out of the signal charge from the DZS 20 by the readout means 30 will be explained.

As shown in the timing chart of FIG. 2, the readout time K of the fluorescent image signal charge obtained by the DZS 20, and the readout time D of the dark noise signal charge component representing an image of the target subject 1 obtained by the DZS 20 when said target subject 1 is not being irradiated by the excitation light are both set at 1/120 seconds.

The subtraction means 40, in which the fluorescent image signal and the dark noise image signal component have been recorded, subtracts the dark noise image signal component from the fluorescent image signal to obtain a subtraction image signal, and outputs said subtraction image signal. The subtraction image signal, which is an observation image signal, outputted from the subtracting means 40 is converted to a visible image signal and displayed on the display 70 at a frame rate of 60 frames/sec.

Note that because the obtainment by the DZS 20 of the image when the excitation light is not being emitted from the light source apparatus 11 as described above is performed within the body cavity of the patient, the dark noise signal charge component obtained by the DZS 20 is substantially the same as the signal charge that represents dark noise, generated when the external light is shielded.

Further, the dark noise image signal component is not necessarily required to be obtained when the fluorescent image is obtained; if the quantity of dark noise mixed with the fluorescent image signal is uniform, said quantity of dark noise can be measured in advance and the value thereof recorded in the subtracting means 40 as the dark noise image signal component.

Still further, as shown in the block drawing of a portion of the fluorescent endoscope apparatus shown in FIG. 3, the fluorescent endoscope apparatus 100 can be further provided with an addition averaging means 60 for addition averaging a plurality of subtraction image signals successively outputted from the subtracting means 40 to obtain an addition averaged subtraction image signal and outputting said addition averaged subtraction image signal, and an observation image signal can be obtained as described below.

A first subtraction image signal obtained by the subtracting means 40 of a first fluorescent image signal and a first dark noise signal outputted from the readout means 30 is recorded in the addition averaging means 60, and then a second subtraction image signal obtained by the subtracting means 40 of a second fluorescent image signal and a second dark noise signal outputted from the readout means 30 is recorded in the addition averaging means 60; the first subtraction image signal and the second subtraction image signal are addition averaged by the addition averaging means 60 to obtain an addition averaged subtraction image signal, and said addition averaged subtraction image signal is output.

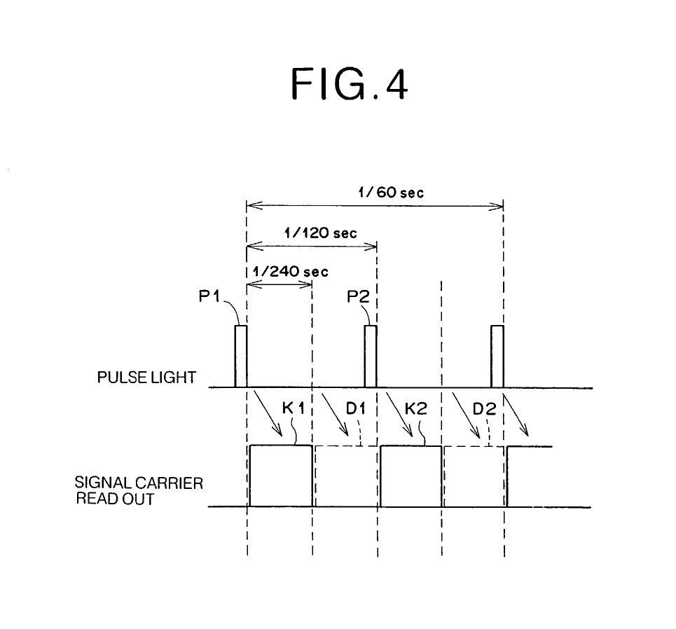

Here, the readout time of reading out the signal charge from the DZS 20 when the addition averaged subtraction image signal is to be formed will be explained using the timing chart shown in FIG. 4.

As shown in the timing chart of FIG. 4, the readout times K1 and K2 of the reading out of the first fluorescent image signal charge and the second fluorescent image signal charge, which have been obtained based on the irradiation of the pulse type excitation lights P1 and P2, from the DZS 20 by the readout means 30, and the readout times D1 and D2 of the reading out by the readout means 30 of the first dark noise signal charge and the second dark noise signal charge obtained when the pulse type excitation light is not being emitted are set at 1/240 seconds (a frame rate of 240 frames/second). Based on the read out fluorescent image signal charges and dark noise signal charge components, a first subtraction image signal and a second subtraction image signal are formed by the subtracting means 40 every 1/120 seconds (each of a frame rate of 120 frames/sec). The two formed and outputted subtraction image signals are then addition averaged by the addition averaging means 60 and outputted as an addition averaged subtraction image signal by the addition averaging means every 1/60 seconds (each of a frame rate of 60 frames/sec).

The subtraction image signal, which is an observation image signal, outputted from the addition averaging means 40 is converted to a visible image signal and displayed on the display 70 at a frame rate of 60 frames/sec.

Note that the setting of the readout time, the control of each operation, and the like are controlled by the controller 50.

Further, as shown in the block drawing of a portion of the fluorescent endoscope apparatus shown in FIG. 5, the fluorescent endoscope apparatus 100 can be further provided with a tissue state computing means 80 instead of the addition averaging means 60; wherein, an observation image signal representing a normalized fluorescent light intensity and an observation image signal representing a fluorescence yield for observing the difference between a tissue in a normal state and a tissue in an abnormal state (a diseased portion) can be obtained as described below.

Note that the fluorescence yield is an indicator for distinguishing between a normal tissue and a diseased tissue based on the fact that the intensity of the autofluorescence emitted, upon the irradiation of a target subject by an excitation light of the same intensity, from a normal tissue is higher than that emitted from a diseased tissue. The fluorescence yield can be represented as the ratio of the intensity of the fluorescent light emitted from the target subject of a body cavity of a patient upon the irradiation thereof by an excitation light to the intensity of the reflected light reflected from the target subject upon the irradiation thereof by a near-infrared light.

Further, the normalized fluorescent light intensity is an indicator for distinguishing between a normal tissue and a diseased tissue based on the fact that the spectral form of the autofluorescence emitted, upon the irradiation of a target subject by an excitation light, from a normal tissue and that emitted from a diseased tissue differ near the 480 nm wavelength range. The normalized fluorescent light intensity can be represented as the ratio of the intensity of the narrow band range fluorescent light component near 480 nm to the to the intensity of the wide band range fluorescent light component spanning from 430-730 nm within the fluorescent light emitted from the target subject of a body cavity of a patient upon the irradiation thereof by the excitation light.

The fluorescence yield and the normalized fluorescent light intensity can be used as stable indicators that are not affected by the interval, angle and/or the like between the emission point of the excitation light and the position of the target subject irradiated by said excitation light.

First, the operation occurring when a fluorescent light yield image signal, which is an observation image signal, representing the fluorescence yield is to be obtained will be explained.

The pulse type excitation light and near-infrared light emitted from the light source apparatus 11 pass through the light guide 12 and are projected onto the target subject 1 at mutually different timings.

After the fluorescent image formed of the fluorescent light emitted from the target subject 1 upon the irradiation thereof by the pulse type excitation light has been detected by the DZS 20 and obtained as a fluorescent image signal charge representing said detected fluorescent image, said fluorescent image signal charge is read out by the readout means 30, and converted to a fluorescent image signal and outputted, and said outputted fluorescent image signal is recorded in the subtracting means 40.

Continuing, while the excitation light is not being emitted from the light source 11, an image of the target subject 1 not irradiated by the excitation light is obtained by the DZS 20, read out by the readout means 30 at the same frame rate as that described above, converted to a dark noise image signal component representing the dark noise, outputted, and recorded in the subtraction means 40.

The subtraction means 40 subtracts the dark noise image signal component from the fluorescent image signal, which has been recorded therein as described above, to obtain a subtraction fluorescent image signal, which is a subtraction image signal, and outputs said subtraction fluorescent image signal to the tissue state computing means 80.

Next, after the near-infrared image formed of the reflected near-infrared light reflected from the target subject 1 upon the irradiation thereof by the pulse type near-infrared light has been detected by the DZS 20 and obtained as a near-infrared image signal charge representing said detected near-infrared image, said near-infrared image signal charge is read out by the readout means 30, converted to a near-infrared image signal and outputted, and recorded in the subtracting means 40 in the same manner as described above.

Continuing, while the excitation light is not being emitted from the light source 11, a dark noise image signal component is recorded in the subtraction means 40 in the same manner as described above.

The subtraction means 40 subtracts the dark noise image signal component from the near-infrared image signal, which has been recorded therein as described above, to obtain a subtraction near-infrared image signal, which is a subtraction image signal, and outputs said subtraction near-infrared image signal to the tissue state computing means 80.

The tissue state computing means 80, in which the subtraction fluorescent image signal and the subtraction near-infrared image signal have been stored, obtains the ratio of the subtraction fluorescent image signal and the subtraction near-infrared image signal to form a fluorescence yield image signal, and outputs said fluorescence yield image signal. The fluorescent light yield rate image signal, which is an observation image signal, outputted from the tissue state computing means 80 is converted to a visible image signal and displayed on the display 70.

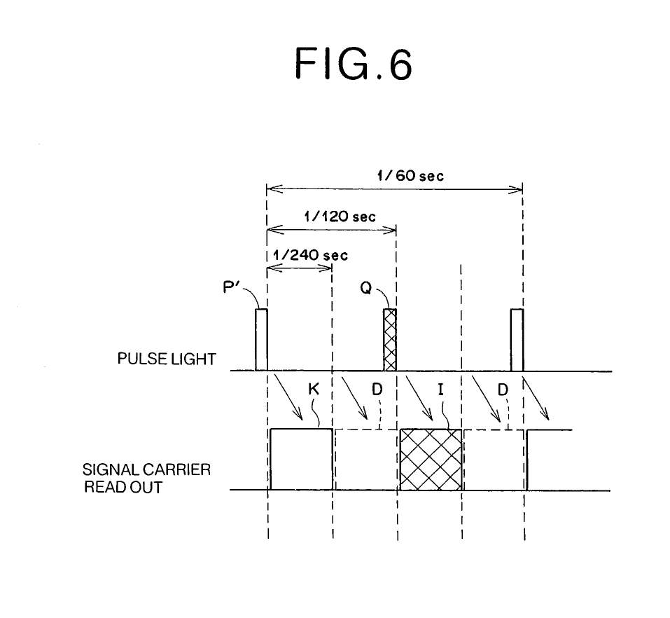

Here, the readout time of the reading out of the signal charge from the DZS 20 when the fluorescence yield is formed will be explained with reference to the timing chart of FIG. 6.

As shown in the timing chart of FIG. 6: the readout time K of the reading out of the fluorescent image signal charge, which has been obtained based on the irradiation of the pulse type excitation light P′, from the DZS 20 by the readout means 30; the readout time D of the reading out by the readout means 30 of the dark noise signal charge obtained when the pulse type excitation light is not being emitted; and the readout time N of the reading out of the near-infrared image signal charge, which has been obtained based on the irradiation of the pulse type near-infrared light Q, are each set at 1/240 seconds (a frame rate of 240 frames/second); and the subtraction fluorescent image signal and the subtraction near-infrared image signal charge are outputted from the subtracting means 40 every 1/120 seconds. Then, the fluorescent yield ratio image signal, which is an observation image signal, is outputted from the tissue state computing means 80 every 1/60 seconds.

Note that the setting of the readout time, the control of each operation, and the like are controlled by the controller 50.

Next, the operation occurring when a normalized fluorescent light intensity image signal, which is an observation image signal representing a normalized fluorescent light intensity, is to be obtained will be explained.

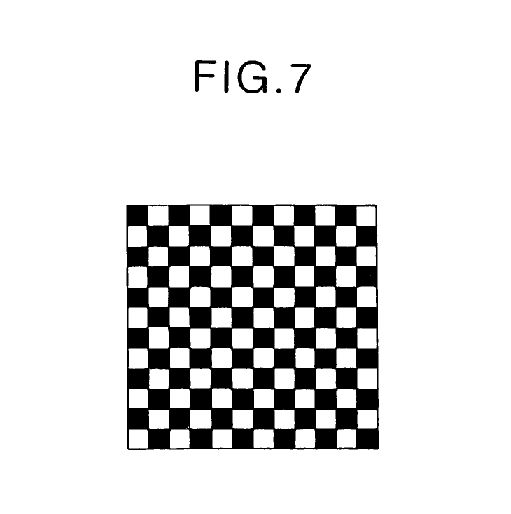

For cases in which a normalized fluorescent light intensity image signal is to be obtained, a mosaic filter 85 provided with two types of filter elements, of which one type of filter element transmits light of a wavelength near the narrow band wavelength range of 480 nm and the other type of filter element transmit light of a wavelength within the wide band wavelength range spanning from 430 to 730 nm, combined in an alternating pattern corresponding to the positions of the light receiving pixels as shown in FIG. 7, is disposed over the light detecting surface 21, and the pulse type excitation light emitted from the light source apparatus 11 passes through the light guide 12 and is projected onto the target subject 1.

The fluorescent light emitted from the target subject 1 upon the irradiation thereof by the pulse type excitation light passes through the mosaic filter 85 and a narrow band fluorescent image that has passed through the narrow band filter elements and a wide band fluorescent image that has passed through the wide band filter elements are simultaneously focused on the light receiving face 21, and obtained as fluorescent image signal charges by the DZS 20. The obtained fluorescent image signal charge is read out by the readout means 30, converted to a fluorescent image signal, and outputted. The outputted fluorescent image signal is recorded in the subtracting means 40.

Continuing, an image of the target subject 1 not being irradiated by the excitation light is obtained by the DZS 20 when the excitation light is not being emitted from the light source apparatus 11, and is read out by the readout means 30 at the same frame rate as described above. The read out image signal charge is converted to a dark noise image signal component and recorded in the subtracting means 40 in the same manner as described above.

The subtracting means 40 subtracts the dark noise image signal component from the fluorescent image signals formed of the simultaneously obtained narrow band fluorescent image and wide band fluorescent image to obtain a subtraction narrow band subtraction image signal corresponding to the narrow band fluorescent image and a subtraction wide band fluorescent image signal corresponding to the wide band fluorescent image, which are subtraction image signals, and outputs said obtained subtraction image signals to the tissue state computing means 80.

The tissue state computing means 80 into which the subtraction narrow band fluorescent image signal and the subtraction wide band fluorescent image signal have been inputted obtains the ratio between said inputted subtraction narrow band fluorescent image signal and subtraction wide band fluorescent image signal to form a normalized fluorescent light intensity image signal, and outputs said normalized fluorescent light intensity image signal. The normalized fluorescent light intensity image signal, which is an observation image signal, outputted from the tissue state computing means 80 is converted to a visible image signal and displayed on the display 70.

Here, the readout time of reading out the signal charge from the DZS 20 when a normalized fluorescent light intensity image is to be formed will be explained.

The readout time of the reading out of the fluorescent image signal charge, which has been obtained based on the irradiation of the target subject by the pulse type excitation light, from the DZS 20 by the readout means 30 and the readout time of the reading out by the readout means 30 of the dark noise signal charge obtained when the pulse type excitation light is not being emitted are both set at 1/240 seconds (a frame rate of 240 frames/second); the subtraction narrow band fluorescent image signal and the subtraction wide band fluorescent image signal charge are outputted simultaneously from the subtracting means 40 every 1/120 seconds. Then, the normalized fluorescent light intensity image signal, which is an observation image signal, is outputted from the tissue state computing means 80 every 1/60 seconds.

Note that the setting of the readout time, the control of each operation, and the like are controlled by the controller 50.

According to the endoscope apparatus employing a charge multiplying shift resistor equipped solid state image obtaining element of the present invention as described above, because an observation image signal is obtained based on the subtraction image signal obtained by subtracting the dark noise image signal component obtained while the target subject is not being irradiated by the excitation light from the image signal obtained when the target subject is being irradiated by the excitation light, the S/N ratio of the observation image signal can be improved compared to that of a conventionally obtained observation image signal.

Note that according to the embodiment described above, although an example wherein a signal charge has been read out from the charge multiplying shift resistor equipped solid state image obtaining element at a frame rate greater than 60 frames/sec has been proffered, even if the signal charge is read out at a frame rate less than or equal to 60 frames/sec, the dark noise contained in the observation image signal is reduced in the same manner as occurs in the above-described embodiment, and the effect whereby the S/N ratio of the observation image signal can be improved compared to that of a conventionally obtained observation image signal can be obtained.

What is claimed is:

1. An image obtaining method for an endoscope, comprising the steps of:
obtaining, by use of a charge multiplying shift resistor equipped solid state image obtaining element, an image formed of the light emitted or reflected from a target subject upon the irradiation thereof by a light, and a signal charge representing the obtained image,
reading out, by use of a readout means, said signal charge and outputting an image signal based on the read out signal charge, and
obtaining an observation image signal based on the outputted image signal, wherein
the dark noise image signal component representing the dark noise included within the image signal read out by the readout means is subtracted from said image signal to obtain the observation image signal.

2. An image obtaining apparatus comprising:
an illuminating means for projecting a light onto a target subject,
a charge multiplying shift resistor equipped solid state image obtaining element for obtaining an image formed of the light emitted or reflected from a target subject upon the irradiation thereof by the aforementioned light, and a signal charge representing the obtained image, and
a readout means for reading out said signal charge from the charge multiplying shift resistor equipped solid state image obtaining element and outputting an image signal based on the read out signal charge, further comprising
a subtracting means for subtracting the dark noise image signal component representing the dark noise included within the image signal read out by the readout means from said image signal to obtain a subtraction image signal.

3. An image obtaining apparatus as defined in claim 2, wherein
said dark noise image signal component is the image signal read out from the charge multiplying shift resistor equipped solid state image obtaining element and output by the readout means when the light is not being emitted.

4. An image obtaining apparatus as defined in claim 2, wherein
said dark noise image signal component is obtained in advance and prerecorded in the subtraction means.

5. An image obtaining apparatus as defined in any of the claims 2, 3, or 4, further comprising
an addition averaging means for adding and averaging a plurality of subtraction image signals successively outputted from the subtraction means to obtain an addition averaged subtraction image.

6. An image obtaining apparatus as defined in any of the claims 2, 3, or 4, wherein
said readout means is a readout means that reads out the signal charge at a frame rate greater than 60 frames/sec.
